(19) United States
(12) Patent Application Publication
(10) Pub. No.: US 2025/0039615 A1
(43) Pub. Date: Jan. 30, 2025

(54) A SOUND RECEIVER WATCH FOR NON-SURGICAL HEARING AID

(71) Applicant: WEHEAR INNOVATIONS PRIVATE LIMITED, Gujarat (IN)

(72) Inventor: Kanishka PATEL, Gujarat (IN)

(21) Appl. No.: 18/716,163

(22) PCT Filed: Nov. 18, 2022
(86) PCT No.: PCT/IB2022/061121

§ 371 Date: Jun. 4, 2024

(30) Foreign Application Priority Data
Jun. 8, 2022 (IN) 202223032861

(51) Int. Cl. H04R 25/00

(52) U.S. Cl. CPC H04R 25/554; H04R 25/558; H04R 25/606; H04R 2460/13

(57) ABSTRACT

The sound receiver watch (100) for non-surgical hearing aid comprising a body detachably connected to the wrist band, said body accommodated with a main circuit powered with the battery (106) placed inside the body. The main circuit having inbuilt sound processor connected to a microphone (102) module being capable to process the sound, a transmitter being capable to transmit said processed sound to said hearing aid device, plurality of buttons to access the functions of said watch. The touch enabled display placed on the top side (105) of said body being connected to a microprocessor and said control circuit, a sort-distance wireless communication module being capable to provide the connectivity to said watch (100) and an USB charging port (103) located on said body, provided to charge said watch (100) battery (106) to operate the same, wherein, watch (100) dissemble as a transmitter and transmit the processed sound to said hearing aid device, said sound being bypass and received to the cochlea directly through the hearing aid device.

This application is the patent of addition of Indian patent application 202021024454 filed on 10 Jun. 2020.

### FIELD OF THE INVENTION

The present invention related to a sound receiver watch for non-surgical hearing aid. More particularly, it is a watch for bone conduction hearing aid device which transmit the processed sound to cochlea directly through the hearing aid device.

### BACKGROUND OF THE INVENTION

Bone-conduction microphone be exactly the bone that utilizes people, skin, muscle etc. as the medium of conduct vibrations, only the vibration plane of its bone-conduction microphone need be close to user's health, as cheek position, the vibration being produced can be passed to people by bone-conduction microphone.

Bone conduction is the conduction of sound to the inner ear primarily through the bones of the skull, allowing the hearer to perceive audio content without blocking the ear canal. Bone conduction transmission occurs constantly as sound waves vibrate bone, specifically the bones in the skull, although it is hard for the average individual to distinguish sound being conveyed through the bone as opposed to the sound being conveyed through the air via the ear canal. Intentional transmission of sound through bone can be used with individuals with normal hearing as with bone-conduction headphones or as a treatment option for certain types of hearing impairment. Bone generally conveys lower-frequency sounds better than higher frequency sounds.

In this vibration transmittance process, greatly weakened the oscillation of sound pressure of transmitting in air, factor affected by environment is very little, and vibration is directly delivered to inner ear and make people hear sound, like this, privacy is better, in noisy environment, also can know and hear sound, therefore, above-mentioned many benefits are expanded the application of bone-conduction microphone greatly.

In recent years, the consumption electronic product of intelligent watch formula is just popular, and a lot of producers have all released the intelligent watch scheme of oneself, and some also possesses call function because the size of intelligent watch is smaller, so the electric elements power of wrist-watch the inside is also relatively little, the sound sending equally can be too small, make sound be difficult to be caught by people, especially for the intelligent watch with call function, because wrist-watch itself is undersized, wrist-watch cannot be used near ear and face as mobile phone simultaneously, so can only adopt hand-free call mode, cause like this dialog context easily by others, to be heard, privacy is difficult to guarantee.

The purpose of the present invention is to provide a kind of wrist wearable type bone conduction sound-conducting apparatus that adopts the transaudient mode of bone conduction, it is applied on intelligent watch, thereby having solved on traditional intelligence wrist-watch technical problem.

CN103135440A provides a Bluetooth smart watch which comprises a watch body and a wireless charger. The Bluetooth module, a wireless charging module, a lithium battery, a small motor, a horn, a microphone, a micro control unit (MCU) and an electrophoretic display (EPD) module are integrated inside the watch body. The wireless charging module is in connection with the lithium battery. The Bluetooth module, the lithium battery, the small motor, the horn, the microphone and the EPD module are respectively connected with the MCU. The wireless charger is connected with the wireless charging module inside the watch body through a wireless electric power transmission technology. A Bluetooth technology is adopted for data interaction between a mobile phone and the watch, a wireless charging technology is utilized for charging the watch, and the watch is small in thickness and multifunctional. Time for watching a message, weather forecast and communication is saved for people, and convenience is brought when the people are in occasions where a call is inconvenient to answer.

U.S. Pat. No. 9,497,307B2 discloses a smart watch with a telephone function module and dial keys. Each dial key corresponds to an unchanged telephone number. After a Bluetooth connection between the smart watch and a primary mobile phone is established, both the Smart watch and the primary mobile phone give a buzzing warning when the Smart watch and the primary mobile phone detect that the Bluetooth connection is interrupted or a Bluetooth signal value is Smaller than a set value. The smart watch is further provided with a temperature sensor, an acceleration sensor and a GPS module, and both the Smart watch and the primary mobile phone give a buzzing warning if the sensors detect when change values of temperature and acceleration exceed present values or if the geographical location of the Smart watch is not within a set range.

CN102866623A an intelligent watch belonging to the field of watch devices. The intelligent watch comprises a time counter, a controller, a display, a timer and a pressure sensor, wherein the time counter is electrically connected with the controller; and the controller is electrically connected with the display respectively through the timer and the pressure sensor. The intelligent watch has the beneficial effects that during use, the time counter and the pressure sensor transmit a time value and a pressure value to the controller constantly; the controller compares the time values and the pressure values; when the pressure value indicates that a user intends to know the time, the controller controls the timer to start to count down and displays the time value on a display screen; when the timer counts down to zero, the controller controls the display to be switched off, so that electric energy is saved; and the intelligent watch is simple in structure and convenient to use.

WO2017062621A1 patent application discloses a smart watch tracking system includes a smart watch and a mobile phone which provide sensor data, such as motion data to a server that analyse and stores the detected sensor information, and provide an associated user interface to the smart watch and mobile phone.

There are many hearing devices available in the market, which are working on conventional methods. Conventional hearing aids are non-invasive (not requiring surgery) and are placed behind the ear, in the canal, or are body-worn. Invasive hearing aids, including bone-anchored hearing aids and cochlear implants, are excluded from coverage in this chapter. Most of the current hearing aids are amplifiers, they work on the principle of amplification where it amplifies the sound level and bombards amplified sound to your eardrums. These hearing aids are not much efficient for people with a higher amount of hearing loss and are not at all useful for people with conductive hearing loss. Another major issue in the current hearing aid is the whistling noise and feedback loop sound. That is mainly because of having a microphone and audio speakers in the same body, which creates feedback noise and create whistling sound. Also, the sound receiver watch for non-surgical hearing aid being suffered from the sound sending too little, during telephone relation, be difficult to guarantee private technical problem.

Conventionally, sound receiver watch for non-surgical hearing aid can necessity to mobile for connection and it is totally depends on mobile or some electronic device connection. It is not self-working as bone conduction hearing aid watch. So, it's depends on wirelessly connection with mobile or some electronic device for working as bone conduction hearing aid device.

The problem associated with the prior technologies, the transmitter and receiver is mounted in one circuit board in sound receiver watch for non-surgical hearing aid, so there are chances to increase the humming, disturbance in the sound and it is difficult to hear to person who have hearing problem. To overcome this problem, the inventors of present inventions came up with new invention which is sound receiver watch for non-surgical hearing aid. The present invention watch act as a transmitter and bone conduction is a receiver, so the transmitter in located in watch and receiver is located in bone conduction hearing aid device, so there are less chance to create any humming, disturbance of whistling due to feedback loop in the sound. The user can clearly recognize the sound by the sound receiver watch for non-surgical hearing aid.

The present invention having receiver that can be used as a hearing aid Bone-conduction hearing aid device with a separate sound processor is useful for people with up to 100% conductive hearing loss, issues in the physical parts of the ear, up to 60 dB SN Hearing loss. The bone conduction hearing device transmits sound in form of vibrations through the cheekbone, which bypasses physical ears and sends sound directly to the cochlea (inner ear), and then it's converted into electrical signals and sent to the brain.

With the present invention, person with any sort of hearing issues due to physical ears can hear. The present invention having a separate ear band or pebble that acts as the sound processor that has microphones and processors inside it. The band captures sound from the surrounding with a microphone system and then this sound is processed through sound-processor, then processed sound with digital signal processing. This sound is sent to devices through Bluetooth, and then with bone conduction, and person can hear surroundings because keeping the sound processor separate it doesn't generate any kind of feedback channel and noise. Also, the present invention does not required any mobile or electronic device for connection. The hearing aid watch can work without any connectivity of mobile or any electronic device. The present invention is more accurate, reliable, compact and user-friendly device.

### OBJECT OF THE INVENTION

The principle object of present invention is to overcome the drawbacks and limitations associated with prior technology by providing a sound receiver watch for non-surgical hearing aid of the same.

Another object of the present invention is to provide a watch dissemble as a transmitter being capable to transmit the processed sound and sound being bypass received to the cochlea directly through the hearing aid device.

Another object of the present invention is to provide a sound receiver watch for non-surgical hearing aid having an inbuilt sound processor connected to a plurality microphone module being capable to process the sound.

Yet another object of the present invention is to provide a sound receiver watch for non-surgical hearing aid having transmitter being capable to transmit the sound in hearing aid device.

One more object of the present invention is to provide a sound receiver watch for non-surgical hearing aid having wireless communication module being capable to provide connectivity to watch.

Yet another object of the present invention is to provide a LED being capable for indication for the function in the sound receiver watch for non-surgical hearing aid.

One more object of the present invention is to provide a sound receiver watch for non-surgical hearing aid which can operate without any electronic device or mobile.

Another object of the present invention is to provide a sound receiver watch for non-surgical hearing aid which is more accurate, reliable, compact and user-friendly device.

Another object of the present invention is to provide a sound receiver watch for non-surgical hearing aid being capable to detach from a wrist band and body independently functioned as the sound processor and transfer said processed sound to said bone conduction hearing aid device.

Another object of the present invention is to provide a plurality of microphone array, which is receiving the sound from 360 degree and sound processor in the watch being capable generate sound for better user experience and noise removal.

### SUMMARY OF THE INVENTION

The main aspect of the present invention is to provide a sound receiver watch for non-surgical hearing aid comprising a body detachably connected to the wrist band, said body accommodated with a main circuit powered with the battery placed inside the body. The main circuit having inbuilt sound processor connected to a plurality microphone module being capable to process the sound, a transmitter being capable to transmit said processed sound to said hearing aid device, plurality of buttons to access the functions of said watch. The touch enabled display placed on the top side of said body being connected to a microprocessor and said control circuit, a short-distance wireless communication module being capable to provide the connectivity to said watch and an USB charging port located on said body, provided to charge said watch battery to operate the same, wherein, watch dissemble as a transmitter and transmit the processed sound to said hearing aid device, said sound being bypass and received to the cochlea directly through the hearing aid device.

As per another aspect of the present invention is to provide a sound receiver watch for non-surgical hearing aid which is bone conduction hearing device which transferred the sound to cochlea without entering in-ear canal. The LED which is indicate the plurality of the functions in the watch.

As per another aspect of the present invention is to provide a sound receiver watch for non-surgical hearing aid being capable to detach from wrist band and body independently functioned as the sound processor and transfer the processed sound to bone conduction hearing aid device.

As per another aspect of the present invention is to provide a plurality of microphone array, which is receiving the sound from 360 degree and sound processor in the watch being capable generate sound for better user experience and noise removal.

### BRIEF DESCRIPTION OF DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific methods and structures disclosed herein. The description of a method step or a structure referenced by a numeral in a drawing is applicable to the description of that method step or structure shown by that same numeral in any subsequent drawing herein.

FIG. 1 is a schematic presentation of the sound receiver watch for non-surgical hearing aid. FIG. 1 shows the microphone placement, switch and PCB in the sound receiver watch for non-surgical hearing aid.

FIG. 2 represents the top side view and internal body structure and arrangement of an embodiment of the sound receiver watch for non-surgical hearing aid. The FIG. 2 shows the microphone placement, USB charging port and battery arrangement in the sound receiver watch for non-surgical hearing aid.

FIG. 3 shows the placement of the switch in said sound receiver watch for non-surgical hearing aid.

FIG. 4 shows the LED indicator, switch and charging port in the said sound receiver watch for non-surgical hearing aid.

### DETAILED DESCRIPTION OF INVENTION

The present invention overcomes the aforesaid drawbacks of the above, and other objects, features and advantages of the present invention will now be described in greater detail. Also, the following description includes various specific details and are to be regarded as merely exemplary. Accordingly, those of ordinary skill in the art will recognize that: without departing from the scope and spirit of the present disclosure and its various embodiments there may be any number of changes and modifications described herein.

It must also be noted that as used herein and in the appended claims, the singular forms “a”, “an,” and “the” include plural references unless the context clearly dictates otherwise. Although any systems and methods similar or equivalent to those described herein can be used in the practice or testing of embodiments of the present invention, the preferred, systems and methods are now described.

As per main embodiment of the present invention is to provide sound receiver watch (100) for non-surgical hearing aid comprising a body detachably connected to the wrist band, said body accommodated with a main circuit powered with the battery (106) placed inside the body;
said main circuit having inbuilt sound processor connected to a plurality microphone (102) module being capable to process the sound, a transmitter being capable to transmit said processed sound to said hearing aid device, plurality of buttons to access the functions of said watch;
An on/off slider switch;
an touch enabled display placed on the top side (105) of said body being connected to a microprocessor and said control circuit;
a sort-distance wireless communication module being capable to provide the connectivity to said watch; and
   an USB charging port (103) located on said body, provided to charge said watch (100) battery (106) to operate the same;
wherein,
said watch (100) dissemble as a transmitter and transmit the processed sound to said hearing aid device, said sound being bypass and received to the cochlea directly through the hearing aid device.

As per another embodiment of the present invention is to provide a main body having inbuilt sound processor connected to a plurality microphone (102) module being capable to process the sound, a transmitter being capable to transmit said processed sound to hearing aid device, plurality of buttons to access the functions of said watch. The audio signal processing is a subfield of signal processing that is concerned with the electronic manipulation of audio signals. The processor captures sound signals and sends them to a receiver implanted under the skin behind the ear. The receiver sends the signals to electrodes implanted in the snail-shaped inner ear (cochlea). The signals stimulate the auditory nerve, which then directs the signals to the brain. The sound processor includes plurality microphone (102) which is receive the sound from the surrounding, for that band have one tiny slot on the top part of the band which is covered with a muffler.

As per one embodiment of the present invention is to provide sound receiver watch (100) for non-surgical hearing aid, the watch (100) dissemble as a transmitter and transmit the processed sound to hearing aid device, the sound being bypass and received to the cochlea directly through the hearing aid device. When the sound come, the watch (100) being capable to transmit the sound to bone conduction hearing aid device and user can hear the sound which is transmitted by watch.

As per one embodiment of the present invention is to provide sound receiver watch (100) for non-surgical hearing aid is able run without any mobile, laptop or electronics device. The watch (100) is directly connect the bone conduction hearing aid device through wirelessly and user can able to hear the sound from the watch (100) through sound processor. The watch (100) is detachable and user can put the watch (100) on desk, table etc. and listen the sound from the bone conduction sound receiver watch (100) for non-surgical hearing aid.

As per one embodiment of the present invention is to provide a slider switch (101) on the one side of the casing is used for turning on or off the Bluetooth mode. The sliding mechanism used for the switch, slot for the switch (101) comes into the main casing of band. The sliding mechanism arrangement of mechanical parts to convert straight-line motion to rotary motion. The watch (100) having a wireless connectivity which is being capable to connect with bone conduction hearing aid device.

As per one embodiment of the present invention is to provide an touch enabled display placed on the top side (105) of said body being connected to a microprocessor and said control circuit. The touch enable display having plurality of function in the said watch (100) for hearing aid device. The touch enable display providing access to all functional features. The sound receiver watch (100) for non-surgical hearing aid which transmit the processed sound to cochlea directly through the hearing aid device aid device and processor for execute the instruction to the memory to store the said watch (100) for hearing aid device.

As per one embodiment of the present invention is to provide a USB charging port (103) which is provided to charge to said watch (100) battery (106) to operate. The sort-distance wireless communication module being capable to provide the connectivity to said watch.

As per one embodiment of the present invention is to provide a sound receiver watch (100) for non-surgical hearing aid, the watch (100) paired with hearing aid device and transmit the processed sound to said hearing aid device, said sound being bypass and received to the cochlea directly through the hearing aid device. There are LED light for indication (104), the LED with Blue and Red LEDs. The red LED indicates (104) the band is in charging and turns off automatically when the band charges 100%. The red LED blinks when the band is in pairing mode. The blue LED indicates band is paired with the device.

As per another embodiment of the present invention is the watch (100) operated as individual sound processor and so reduce the effects of noise. The sound receiver watch (100) for non-surgical hearing aid as claimed in claim 1, wherein said body being capable to detach from a wrist band, wherein said body independently functioned as the sound processor and transfer said processed sound to said bone conduction hearing aid device.

As per another embodiment of the present invention is to provide a sound receiver watch (100) for non-surgical hearing aid, the PCB (107) has PCB (107) having multiple component in the said device.

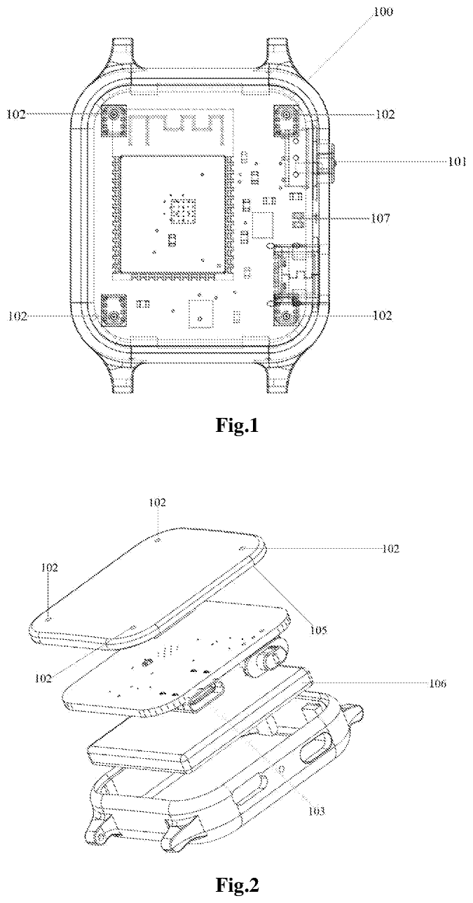

Referring FIG. 1 and FIG. 2 shows the sound receiver watch (100) for non-surgical hearing aid, the On/Off Slider Switch: Slider Switch (101) on the one side of the casing is used for turning on or off the Bluetooth mode. The plurality of microphones (102) and processors inside it. The plurality of microphone (102) being capable to receive the sound from the surrounding, for that band have one tiny slot on the top part of the band which is covered with a muffler. The central processing unit (CPU), also called a central processor is the electronic circuitry that executes instructions in the said sound receiver watch (100) for non-surgical hearing aid. The USB port (103) is a charging slots.

Continuously referring FIG. 1 and FIG. 2 the plurality of microphones (102) placed in a way that sound is captured from 360 degrees and sound processor processes it in a way that user gets perfect surround sound experience. The microphone (102) in watch (100) in generate the sound and which is noise removal. The main part and battery (106) in the sound receiver watch (100) for non-surgical hearing aid. The main part is the cover body which includes all the assembly and it includes battery (106), PCB (107) and Press-fit with the top side part (105) mechanically. The rechargeable battery (106) which is power input and USB charging slot (103) in the main casing of the band.

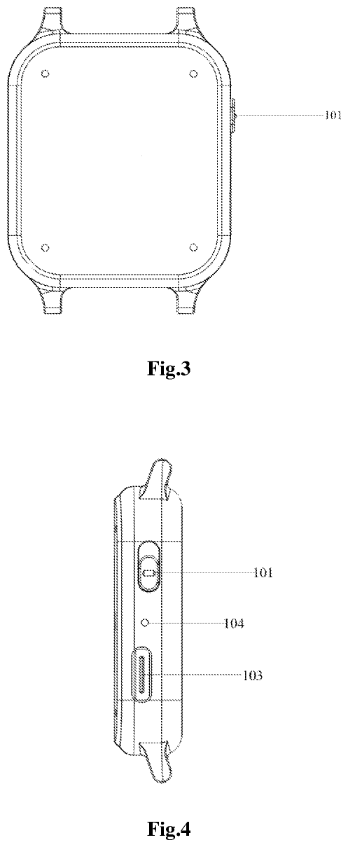

Referring FIG. 3 and FIG. 4 shows the switch (101) in sound receiver watch (100) for non-surgical hearing aid. The On/Off Slider Switch: Slider Switch (101) on the one side of the casing is used for turning on or off the Bluetooth mode. The LED for indication (104) of the said device. The LED indicator (104) with Blue and Red LEDs. The red LED indicates (104) the band is in charging and turns off automatically when the band charges 100%. The red LED blinks when the band is in pairing mode and the blue LED indicates (104) band is paired with the sound receiver watch (100) for non-surgical hearing aid.

Without further description, it is believed that one of ordinary skill in the art can, using the preceding description and the illustrative examples, make and utilize the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

### REFERENCE NUMERALS

Watch 100
Switch 101
Microphone 102
USB Charging Port 103
LED indicator 104
Top Side 105
Battery 106
PCB 107

We claims:

1. A sound receiver watch (100) for non-surgical hearing aid comprising a body detachably connected to the wrist band, said body accommodated with a main circuit powered with the battery (106) placed inside the body;
said main circuit having inbuilt sound processor connected to a plurality of microphone (102) module being capable to process the sound, a transmitter being capable to transmit said processed sound to said hearing aid device, plurality of buttons to access the functions of said watch;
an on/off slider switch (101);
an touch enabled display placed on the top side (105) of said body being connected to a microprocessor and said control circuit;
a sort-distance wireless communication module being capable to provide the connectivity to said watch; and
an USB charging port (103) located on said body, provided to charge said watch (100) battery (106) to operate the same;
wherein,
said watch (100) dissemble as a transmitter and transmit the processed sound to said hearing aid device, said sound being bypass and received to the cochlea directly through the hearing aid device.

2. The sound receiver watch (100) for non-surgical hearing aid as claimed in claim 1, wherein said band compatible with said hearing aid device which is bone conduction hearing aid device which transferred the sound to cochlea without entering in-ear canal.

3. The sound receiver watch (100) for non-surgical hearing aid as claimed in claim 1, wherein said watch (100) further comprising at least one LED indicator (104) provided as indicator for the plurality of functions of said watch.

4. The sound receiver watch (100) for non-surgical hearing aid as claimed in claim 1, wherein said watch (100) operated as individual sound processor and so reduce the effects of noise.

5. The sound receiver watch (100) for non-surgical hearing aid as claimed in claim 1, wherein said body being capable to detach from a wrist band, wherein said body independently functioned as the sound processor and transfer said processed sound to said bone conduction hearing aid device.

6. The sound receiver watch (100) for non-surgical hearing aid as claimed in claim 1, wherein said plurality of microphone (102) array being capable to receive the sound from 360 degree and said sound in the watch being capable to generate sound for better user experience and noise removal.
